(19) United States
(12) Patent Application Publication
(10) Pub. No.: US 2005/0215781 A1
(43) Pub. Date: Sep. 29, 2005

(54) Novel polymorph of cefdinir

(75) Inventors: Ramasubbu Chandrasekaran, Chennai (IN); Krishnan Senthilkumar, Dindigul (IN); Saravan Murugan, Chennai (IN); Venkatasubba Raju Sivaiah Sangaraju, Chennai (IN); Gaddam Om Reddy, Chennai (IN)

Correspondence Address: OLIFF & BERRIDGE, PLC, P.O. BOX 19928, ALEXANDRIA, VA, 22320, US

(73) Assignee: ORCHID CHEMICALS & PHARMACEUTICALS LTD., Chennai (IN)

(21) Appl. No.: 11/079,180

(22) Filed: Mar. 15, 2005

Related U.S. Application Data
(60) Provisional application No. 60/553,552, filed on Mar. 17, 2004.

(51) Int. Cl. C07D 501/12

(52) U.S. Cl. 540/222

(57) ABSTRACT

The present invention relates to novel polymorph of cefdinir represented by formula (I). More particularly, the present invention relates to novel crystalline form (Crystal D) of cefdinir. 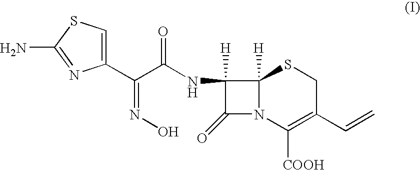 The present invention also provides a process for the preparation of novel crystalline form (Crystal D) of cefdinir.

### FIELD OF THE INVENTION

The present invention relates to novel polymorph of cefdinir represented by formula (I). More particularly, the present invention relates to novel crystalline Form D of cefdinir. 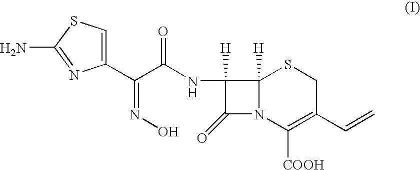
The present invention also provides a process for preparing novel crystalline Form D of cefdinir.

### BACKGROUND OF THE INVENTION

Cefdinir is a third generation cephalosporin antibiotic for oral administration and has a. broader antibacterial spectrum over the general gram positive and gram negative bacteria, especially against Streptococci, than other antibiotics for oral administration.

Cefdinir is a very useful anti microbial agent and was first time described in U.S. Pat. No. 4,559,334. The U.S. Pat. No. 4,935,507 disclosed that cefdinir reported in U.S. Pat. No. 4,559,334 is a crystalline like amorphous product, not a crystalline product and has disadvantages like it is bulky, not so pure, unstable and insufficient in filtration rate, therefore it is not suitable for a pharmaceutical product or is not easy to handle in the pharmaceutical preparations, in producing it in a large scale or in storage.

U.S. Pat. No. 4,935,507 claims the novel crystalline Form (Crystal A) of the cefdinir and a process for preparing the same. The X-ray crystallography data given in this patent is as given in the following table:

| 2 θ° | I % |
|---|---|
| 14.7 | 76 |
| 17.8 | 56 |
| 21.5 | 100 |
| 22.0 | 70 |
| 23.4 | 38 |
| 24.4 | 80 |
| 28.0 | 40 |

The crystalline Form (Crystal A) of cefdinir is disclosed in U.S. Pat. No. 4,935,507 is prepared by dissolving 7-[2-(2-aminothiazol-4-yl)-2-hydroxyiminoacetamido]-3-vinyl-3-cephem-4-carboxylic acid (syn isomer)(an amorphous product) as a salt with alkali metal in a solvent and adjusting the pH at room temperature.

US patent application publication no 2003/0204082 discloses a new crystalline Form which has high stability but a dissolution rate less than that of crystal A.

In view of the vital antibiotic activities of cefdinir of the formula (I), the inventors of the present invention succeeded in obtaining the compound (I) in novel crystal Form, i.e. Crystal D, by adjusting the pH of a solution of cefdinir salt in mixture of water and organic solvent to 2.5 to 2.7 at low temperatures to get cefdinir with new crystal lattice which has better stability.

### OBJECTIVES OF THE INVENTION

The main objective of the present invention is to provide novel crystalline Form D of cefdinir.

Another objective of the present invention is to provide novel crystalline Form D of cefdinir, which has better stability and useful for developing different dosage forms.

Another objective of the present invention is to provide process for novel crystalline Form D of cefdinir

### SUMMARY OF THE INVENTION

Accordingly, the present invention provides novel crystalline Form D of cefdinir of the formula (I) 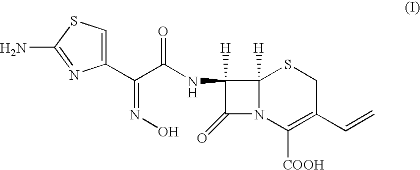
having substantially the same X-ray diffractogram as set out in FIG. 1, and substantially the same IR spectrum, in a KBr, as set out in FIG. 2.

The present invention also provides a process for the preparation of novel crystalline Form D of cefdinir of the formula (I), which comprises the steps as per the scheme: 1

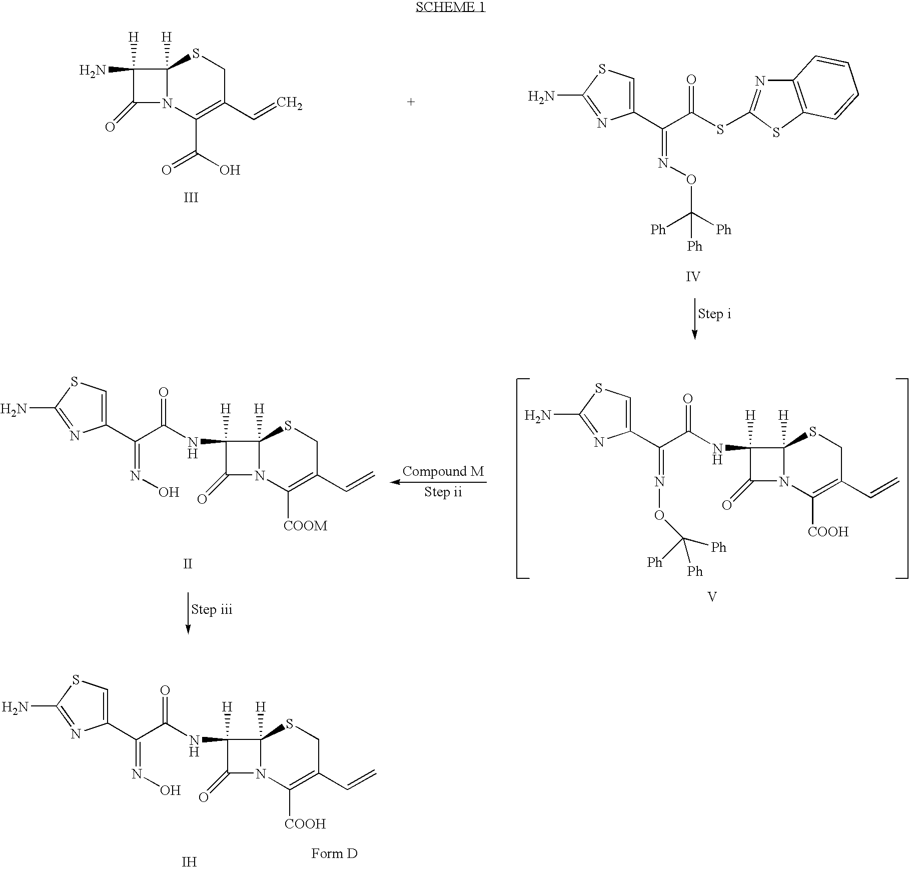

An embodiment of the present invention provides a process for the preparation of compound of formula (II), which comprising the steps of:
i) condensing 7-amino-3-cephem-4-carboxylic acid of the formula (III) with compound of the formula (IV) in the presence of a base and solvent to give trityl intermediate of formula (V)
ii) hydrolyzing the trityl intermediate of formula (V) insitu with an acid in the temperature range of 35-75° C. to give freebase, which is treated with compound M to produce a compound of formula (II) where in compound M is group which forms salt with Cefdinir in mixture of organic solvent and water
iii) converting the compound of formula (II) into Cefdinir Form D.

In another embodiment of the present invention, provides a process for the preparation of Form D from the compound of formula (II), which comprising the steps of:
a) dissolving the compound of formula (II) in mixture of solvents, acidifying the solution to pH 0.5-2.0 preferably at 1.3 to 1.5 at a temperature in the range of 10 to 50° C. and preferably at 15 to 36° C.
b) adjusting the pH of solution obtained from step (a) to 4.0-6.5 using a base and treating with charcoal at a temperature in the range of 10-50° C. preferably at 15 to 36° C. and filtered
c) readjusting the pH of filtrate obtained from step (b) to 2.0-3.0 preferably to 2.5 to 2.7 using acid at a temperature in the range of 10-50° C. preferably at 15 to 36° C.
d) isolating Form D of cefdinir of the formula (I) from step (c)

### DESCRIPTION OF FIGURES

FIG. 1: Powder XRD pattern of crystalline Form D of cefdinir of formula (I).

FIG. 2: Infrared Spectrum of crystalline Form D of cefdinir of formula (I).

### DETAILED DESCRIPTION OF THE INVENTION

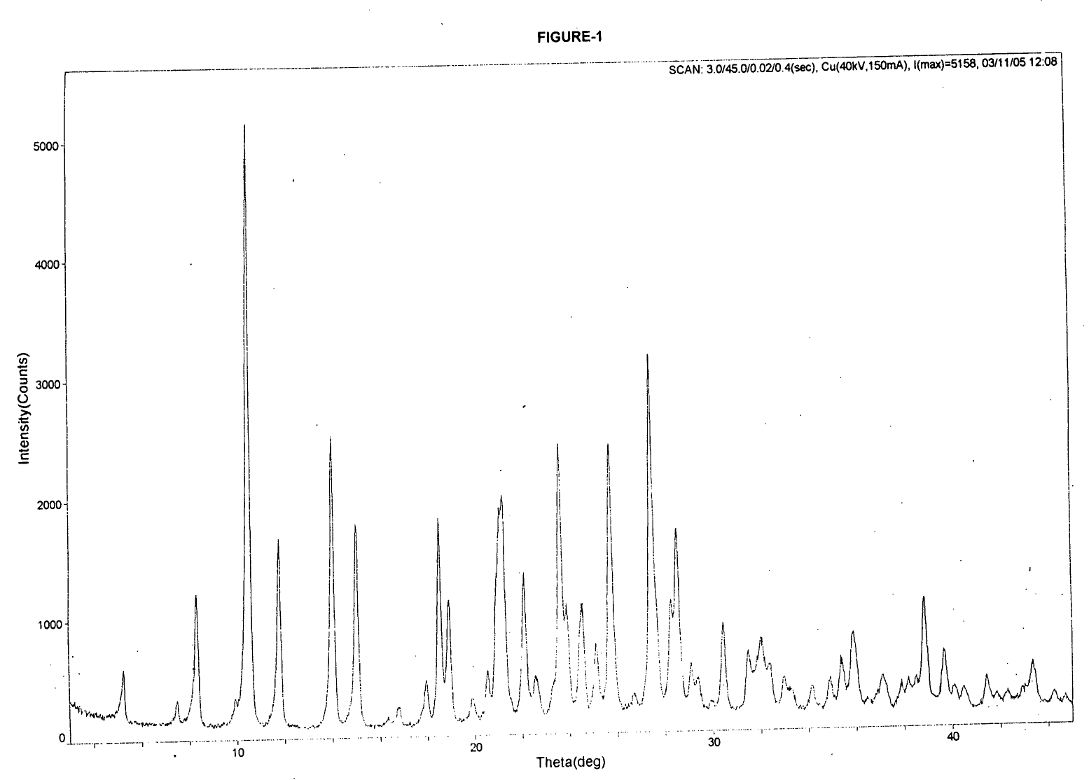

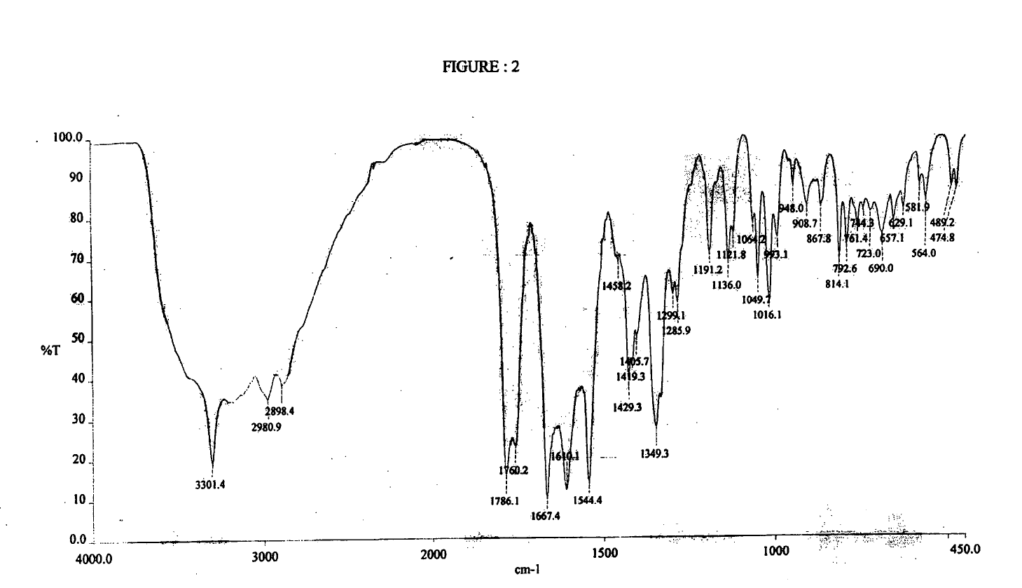

The present invention provides novel crystalline Form D of cefdinir of the formula (I) 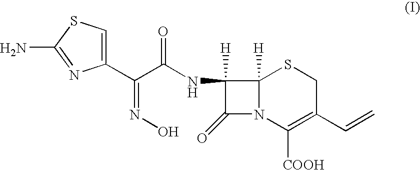
having substantially the same X-ray diffractogram as set out in FIG. 1, and substantially the same IR spectrum, in a KBr, as set out in FIG. 2

In an embodiment of the present invention, novel crystalline Form D of cefdinir of the formula (I) is characterized by X-ray powder diffraction pattern with characteristic peaks of following 2θ values

| 2-Theta | I/Iₒ |
|---|---|
| 5.301 | 8.36 |
| 8.399 | 8.27 |
| 10.619 | 100 |
| 11.859 | 18.99 |
| 14.101 | 35.03 |
| 15.119 | 12.74 |
| 18.6 | 10.67 |
| 18.98 | 6.97 |
| 21.32 | 24.79 |
| 22.18 | 8.23 |
| 23.701 | 21.46 |
| 23.96 | 12.33 |
| 24.6 | 8.18 |
| 25.819 | 15.17 |
| 27.54 | 32.7 |
| 28.36 | 13.89 |
| 28.6 | 17.75 |
| 29.161 | 7.88 |
| 30.519 | 6.15 |
| 32.18 | 5.85 |
| 35.94 | 7.62 |
| 38.94 | 7.21 |

In another embodiment of the present invention, step (i) of condensing compound of formula III with compound of formula IV in the presence of base selected from triethylamine, N-methylpiperidine, N,N-diisopropylethylamine or trimethylamine, pyridine, morpholine, piperidine, aniline triethylamine, diethylamine, tributylamine, pyridine, N-alkylanilines, N-methylmorpholine or mixtures thereof, preferably in triethylamine and solvent selected from ethanol, methanol, isopropanol, Tetrahydrofuran (THF), acetone, butan-2-one, acetonitrile, dioxane, N,N-dimethylformamide (DMF), N,N′dimethylacetamide (DMAc), water or a mixture thereof preferably mixture of water and THF

In an embodiment of the present invention, in step (ii) compound M is selected from ammonium hydroxide, dicyclohexylamine, N,N′-dibenzyl ethylenediamine, 1,8-diazabicyclo(5.4.0)undec-7-ene(DBU), 1,5-diazabicyclo (4.3.0)non-5-ene, N,N′-dicyclohexylethylenediamine (DDA), N,N′-diphenyl ethylenediamine, 1,4-dizabicyclo(2.2.2)octane, N,N-diisopropylethylamine, N,N-diisopropylethylamine and the like preferably N,N′-dicyclohexylethylenediamine (DDA)

In another embodiment of the present invention, acid selected for hydrolyzing the trityl group is selected from an inorganic acid such as hydrochloric acid, hydrobromicacid, hydroiodic acid, sulphuric acid, lewis acid such as aluminum chloride, boron triflouride, boron trifluoride etherate and the like, an organic acid such as acetic acid, formic acid, trifluoroacetic acid, methanesulfonic acid, benzenesulphonic acid, p-toluenesulfonic aicd, and the like preferably hydrochloric acid.

In another embodiment of the present invention, N,N′-dicyclo hexylethylenediamine (DDA) salt obtained from step (ii) is crystalline

In another embodiment of the present invention, cefdinir salt obtained step (ii) or compound of formula II is converted to Cefdinir Form D by dissolving in sole selected from C₁₋₄ alcohol, acetone, acetonitrile, THF, Dimethylformamide (DMF), Dimethylacetamide (DMAc), dimethylsulfoxide (DMSO), ethyl acetate, and water or their mixtures thereof preferably in the mixture of water and acetone

In another embodiment of the present invention, solution obtained from step (iii; (a)) pH is adjusted to 4.0-6.5 using alkali metal hydroxides like sodium hydroxide, potassium hydroxide, alkali metal carbonates and bicarbonates like sodium carbonate, potassium carbonate, potassium bicarbonate, sodium bicarbonate and ammonia preferably with ammonia solution and treating with charcoal at a temperature in the range of 10-50° C. preferably at 15 to 36° C. and filtered.

In yet another embodiment of the present invention, the crystalline form (Crystal D) of cefdinir obtained is having moisture content 13-18%, preferably 15%.

In still another embodiment of the present invention the pH of the filtrate obtained from step (iii; (b)) is readjusted to 2.5 to 2.7 at a temperature in the range of 10-50° C. preferably at 15-36° C.

In yet another embodiment of the present invention, the compound of formula (I) obtained is a syn isomer.

The foregoing technique has been found to be markedly attractive, both from commercial point of view, as well as from manufacturing viewpoint and affords good quality of Cefdinir Form D of the formula (I).

Many other beneficial results can be obtained by applying disclosed invention in a different manner or by modifying the invention with the scope of disclosure.

The present invention is provided by the examples given below, which are provided by way of illustration only and should not be considered to limit the scope of the invention.

### EXAMPLE 1

### Preparation of N,N′-dicyclohexylethane-1,2-diamine salt of 7β-[2-(2-amino-4-thiazolyl)-2-(Z-hydroxyimino)acetamido]-3-vinyl-3-cephem-4-carboxylic acid from 7-amino-3-vinyl-3-cephem-4-carboxylic acid (Cefdinir DDA salt)

To a chilled suspension of 7-amino-3-vinyl-3-cephem-4-carboxylic acid (100 gm) in a mixture of tetrahydrofuran (500 mL) and water (62.5 mL), triethylamine (90 gm) was added at 20±2° C. 2-Mercaptobenzothiazolyl (Z)-(2-aminothiazol-4-yl)-2-(trityloxyimino)acetate (260 gm) was added and was stirred at 32±2° C. for 4-6 hours. The reaction was monitored by HPLC. After completion of the reaction, the THF was distilled off to get residue. To the residue, acetone (600 mL) and aqueous Hydrochloric acid (400 mL) were added and heated to reflux and maintained for 35 minutes then chilled acetone (3600 mL) was added and pH was adjusted to 2.0-2.5 with triethylamine. A solution of N,N′-dicyclohexylethane-1,2-diamine in isopropyl alcohol (80 gm in 200 mL) was added to the filtrate and stirred for 60 minutes. The product thus obtained was filtered, washed with acetone and dried to get the title compound 220 gm [HPLC purity 98.27% and water content 1.0%].

### EXAMPLE 2

Preparation of N,N′-dicyclohexylethane-1,2-diamine salt of 7β-[2-(2-amino-4-thiazolyl)-2-(Z-hydroxyimino)acetamido]-3-vinyl-3-cephem-4-carboxylic acid from 7-amino-3-vinyl-3-cephem-4-carboxylic acid

To a chilled suspension of 7-amino-3-vinyl-3-cephem-4-carboxylic acid (50 gm) in a mixture of tetrahydrofuran (300 ml) and water (37.5 ml), triethylamine (45 gm) was added at 20±2° C. 2-Mercaptobenzothiazolyl (Z)-(2-aminothiazol-4-yl)-2-(trityloxyimino)acetate (130 gm) was added and was stirred at 32±2° C. for 4-6 hours. The reaction was monitored by HPLC. After completion of the reaction, the THF was distilled off to get residue. To the residue acetone (300 ml) and aqueous Hydrochloric acid (200 ml) were added and heated to reflux and maintained for 35 minutes then chilled acetone (1800 ml) added and pH was adjusted to 2.0-2.5 with ammonia solution. A solution of N,N′-dicyclohexylethane-1,2-diamine in a mixture acetone and methanol (40 gm in 200 ml; (1:1)) was added to the filtrate to adjust the pH of the solution to 5.5-5.75 and stirred. The product thus obtained was filtered, washed with acetone and dried to get the title compound 135.5 gm [HPLC purity 97.11% and water content 1.66%].

### EXAMPLE: 3

### Preparation of N,N′-dicyclohexylethane-1,2-diamine salt of 7β-[2-(2-amino-4-thiazolyl)-2-(Z-hydroxyimino)acetamido]-3-vinyl-3-cephem-4-carboxylic acid from 7-amino-3-vinyl-3-cephem-4-carboxylic acid

To a chilled suspension of 7-amino-3-vinyl-3-cephem-4-carboxylic acid (10 gm) in a mixture of DMAc (60 ml) and water (7.5 ml), triethylamine (9.0 gm) was added at 20±2° C. 2-mercaptobenzothiazolyl (Z)-(2-aminothiazol-4-yl)-2-(trityloxyimino) acetate (26 gm) was added and was stirred at 32±2° C. for 4-6 hours. The reaction was monitored by HPLC. After completion of the reaction, acetone (40 ml) and aqueous Hydrochloric acid (43 ml) were added and reaction mixture was heated to reflux for 2 hours. The reaction was monitored by HPLC. Acetone (500 ml) was added and pH was adjusted to 2.5 with ammonia. A methanolic solution of N,N′-dicyclohexylethane-1,2-diamine (8 gm in 25 ml) was added to the filtrate to adjust the pH of the solution to 5.5-5.75 and stirred for 30 minutes. The product thus obtained was filtered, washed with acetone and dried to get title compound 23.1 gm [HPLC purity 98.77% and water content 2.46%].

### EXAMPLE: 4

### Preparation of N,N′-dicyclohexylethane-1,2-diamine salt of 7β-[2-(2-amino-4-thiazolyl)-2-(Z-hydroxyimino)acetamido]-3-vinyl-3-cephem-4-carboxylic acid from 7-amino-3-vinyl-3-cephem-4-carboxylic acid

To a chilled suspension of 7-amino-3-vinyl-3-cephem-4-carboxylic acid (10 gm) in a mixture of DMF (60 ml) and water (7.5 ml), triethylamine (9.0 gm) was added at 20±2° C. 2-mercaptobenzothiazolyl (Z)-(2-aminothiazol-4-yl)-2-(trityloxyimino) acetate (26 gm) was added and was stirred at 32±2° C. for 4-6 hours. The reaction was monitored by HPLC. After completion of the reaction, acetone (40 ml) and aqueous hydrochloric acid (43 ml) were added and reaction mixture was heated to reflux for 2 hours. The reaction was monitored by HPLC. Acetone (500 ml) was added and pH was adjusted to 2.5 with ammonia. A methanolic solution of N,N′-dicyclohexylethane-1,2-diamine (8 gm in 25 ml) was added to the filtrate to adjust the pH of the solution to 5.5-5.75 and stirred for 30 minutes. The product thus obtained was filtered, washed with acetone and dried to get the title compound 23.1 gm [HPLC purity 98% and water content 1.00%].

### EXAMPLE 5

### Preparation of 7-[[(2-aminothiazol-4-yl)-(z)(hydroxyimino)]acetyl]amino-3-vinyl-3-cephem-4-carboxylic acid (Form D) from Cefdinir DDA Salt

Cefdinir DDA salt (125g) is stirred in a mixture of water (3750 mL) and acetone (250 mL) at 35 to 38° C. Aqueous Hydrochloric acid is added to adjust pH to 1.2-1.8. After stirring for 5 to 10 minutes, pH is adjusted to 6.0 using ammonia solution, (100 mL). Then carbon is added and stirred at 35 to 38° C. for 30 minutes. The filtrate is cooled to 15° C. and pH is adjusted to 1.5 using aqueous Hydrochloric acid to get a clear solution. Then pH is readjusted to 2.5 using ammonia solution at 10 to 15° C. The white slurry is stirred for 3 hours. Then the precipitate is filtered, washed with water and is air dried to get Cefdinir D 66.5 g [HPLC assay 98-99% (OAB), water content 15.07%,]

### EXAMPLE 6

### Preparation of 7-[[(2-aminothiazol-4-yl)-(z)(hydroxyimino)]acetyl]amino-3-vinyl-3-cephem-4-carboxylic acid (Form D) from Cefdinir DDA Salt

Cefdinir DDA salt (50 g) is stirred in a mixture of water (1500 mL) and Methanol (100 mL) at 35 to 38° C. Aqueous Hydrochloric acid is added to adjust pH to 1.2-1.8. After stirring for 5 to 10 minutes, pH is adjusted to 6.0 using ammonia solution (55 mL). Then carbon is added and stirred at 35 to 38° C. for 30 minutes. The filtrate, is cooled to 15° C. and pH is adjusted to 1.5 using aqueous Hydrochloric acid to get a clear solution. Then pH is readjusted to 2.5 using ammonia solution at 10 to 15° C. The white slurry is stirred for 3 hours. Then the precipitate is filtered, washed with water and is air dried to get Cefdinir D: 26.5 g [HPLC assay 99-99.5%, water content 14.49%]

### EXAMPLE 7

### Preparation of 7-[[(2-aminothiazol-4-yl)-(z)(hydroxyimino)]acetyl]amino-3-vinyl-3-cephem-4-carboxylic acid (Form D) from Cefdinir DDA Salt

Cefdinir DDA salt (50 g) is stirred in a mixture of water (1500 mL) and acetonitrile (100 mL) at 35 to 38° C. Aqueous Hydrochloric acid is added to adjust pH to 1.2-1.8. After stirring for 5 to 10 minutes, pH is adjusted to 6.0 using ammonia solution (55 mL). Then carbon is added and stirred at 35 to 38° C. for 30 minutes. The filtrate is cooled to 15° C. and pH is adjusted to 1.5 using aqueous Hydrochloric acid to get a clear solution. Then pH is readjusted to 2.5 using ammonia solution at 10 to 15° C. The white slurry is stirred for 3 hours. Then the precipitate is filtered, washed with water and is air dried to get Cefdinir D: 26.4 g [HPLC assay 99-99.5%, water content 14.69%]

### EXAMPLE 8

Cefdinir DDA salt (50 g) is stirred in a mixture of water (1500 mL) and isopropyl alcohol (100 mL) at 35 to 38° C. Aqueous Hydrochloric acid is added to adjust pH to 1.2-1.8. After stirring for 5 to 10 minutes, pH is adjusted to 6.0 using ammonia, solution (55 mL). Then carbon is added and stirred at 35 to 38° C. for 30 minutes. The filtrate is cooled to 15° C. and pH is adjusted to 1.5 using aqueous Hydrochloric acid to get a clear solution. Then pH is readjusted to 2.5 using ammonia solution at 10 to 15° C. The white slurry is stirred for 3 hours. Then the precipitate is filtered, washed with water and dried to get Cefdinir D: 26.5 g [HPLC assay 99-99.5%, water content 14.84%]

### EXAMPLE 9

### Preparation of 7-[[(2-aminothiazol-4-yl)-(z)(hydroxyimino)]acetyl]amino-3-vinyl-3-cephem-4-carboxylic acid (Form D) from Cefdinir DDA Salt

Cefdinir DDA salt (50 g) is stirred in a mixture of water (1500 mL) and dimethylformamide (100 mL) at 35 to 38° C. Aqueous Hydrochloric acid is added to adjust pH to 1.2-1.8. After stirring for 5 to 10 minutes, pH is adjusted to 6.0 using ammonia solution (55 mL). Then carbon is added and stirred at 35 to 38° C. for 30 minutes. The filtrate is cooled to 15° C. and pH is adjusted to 1.5 using aqueous Hydrochloric acid to get a clear solution. Then pH is readjusted to 2.5 using ammonia solution at 10 to 15° C. The white slurry is stirred for 3 hours. Then the precipitate is filtered, washed with water and is air dried to get Cefdinir D: 27.08 g [HPLC assay 99-99.5%, water content 14.75%]

### EXAMPLE 10

### Preparation of 7-[[(2-aminothiazol-4-yl)-(z)(hydroxyimino)]acetyl]amino-3-vinyl-3-cephem-4-carboxylic acid (Form D) from Cefdinir DDA Salt

Cefdinir DDA salt (25 g) is stirred in a mixture of water (750 mL) and dimethylsulfoxide (50 mL) at 35 to 38° C. Aqueous Hydrochloric acid is added to adjust pH to 1.2-1.8. After stirring for 5 to 10 minutes, pH is adjusted to 6.0 using ammonia solution (27.5 mL). Then carbon is added and stirred at 35 to 38° C. for 30 minutes. The filtrate is cooled to 15° C. and pH is adjusted to 1.5 using aqueous Hydrochloric acid to get a clear solution. Then pH is readjusted to 2.5 using ammonia solution at 10 to 15° C. The white slurry is stirred for 3 hours. Then the precipitate is filtered, washed with water and is air dried to get Cefdinir D: 13.2 g [HPLC assay 99-99.5%, water content 14.52%]

### EXAMPLE 11

### Preparation of 7-[[(2-aminothiazol-4-yl)-(z)(hydroxyimino)]acetyl]amino-3-vinyl-3-cephem-4-carboxylic acid (Form D) from Cefdinir DDA Salt

Cefdinir DDA salt (25 g) is stirred in a mixture of water (750 mL) and Ethylacetate (50 mL) at 35 to 38° C. Aqueous Hydrochloric acid is added to adjust pH to 1.2-1.8. After stirring for 5 to 10 minutes, pH is adjusted to 6.0 using ammonia solution (27.5 mL). Then carbon is added and stirred at 35 to 38° C. for 30 minutes. The filtrate is cooled to 15° C. and pH is adjusted to 1.5 using aqueous Hydrochloric acid to get a clear solution. Then pH is readjusted to 2.5 using ammonia solution at 10 to 15° C. The white slurry is stirred for 3 hours. Then the precipitate is filtered, washed with water and is air dried to get Cefdinir D: 13.2 g [HPLC assay 99-99.5%, water content 14.52%]

### EXAMPLE 12

### Preparation of 7-[[(2-aminothiazol-4-yl)-(z)(hydroxyimino)]acetyl]amino-3-vinyl-3-cephem-4-carboxylic acid (Form D) from Cefdinir DDA Salt

Cefdinir DDA salt (10 g) is stirred in a mixture of solvent of water (150 mL) and acetone (20 mL) at 35 to 38° C. Hydrochloric acid is added to adjust pH to 1.5. After stirring for 5 to 10 minutes, pH is adjusted to 6.0 using sodium hydroxide solution. Then carbon is added and stirred at 35 to 38° C. for 30 minutes. The filtrate is cooled to 15° C. and pH is adjusted to 1.5 using hydrochloric acid. Then pH is readjusted to 2.5 using sodium hydroxide solution at 10 to 15° C. After precipitation of the product, the white slurry is stirred for 3 hours at 5 to 10° C. Then the precipitate is filtered and is air dried to get Cefdinir D 5.2 g. [HPLC assay 99-99.4%, water content 14.39%]

### EXAMPLE 13

### Preparation of 7-[[(2-aminothiazol-4-yl)-(z)(hydroxyimino)]acetyl]amino-3-vinyl-3-cephem-4-carboxylic acid (Form D) from Cefdinir DDA Salt

Cefdinir DDA salt (25 g) is stirred in a mixture of solvent of water (750 mL) and acetone (50 mL) at 35 to 38° C. Hydrochloric acid is added to adjust pH to 1.5. After stirring for 5 to 10 minutes, pH is adjusted to 6.0 using sodium bicarbonate solution. Then carbon is added and stirred at 35 to 38° C. for 30 minutes. The filtrate is cooled to 15° C. and pH is adjusted to 1.5 using hydrochloric acid. Then pH is readjusted to 2.5 using sodium hydroxide solution at 10 to 15° C. After precipitation of the product, the white slurry is stirred for 3 hours at 5 to 10° C. Then the precipitate is filtered and is air dried to get Cefdinir D 13.6 g. [HPLC assay 99-99.5%, water content 14.28%]

### EXAMPLE 14

### Preparation of 7-[[(2-aminothiazol-4-yl)-(z)(hydroxyimino)]acetyl]amino-3-vinyl-3-cephem-4-carboxylic acid (Form D) from Cefdinir DDA Salt

Cefdinir DDA salt (25 g) is stirred in a mixture of solvent of water (750 mL) and acetone (50 mL) at 35 to 38° C. Hydrochloric acid is added to adjust pH to 1.5. After stirring for 5 to 10 minutes, pH is adjusted to 6.0 using potassium carbonate solution. Then carbon is added and stirred at 35 to 38° C. for 30 minutes. The filtrate is cooled to 15° C. and pH is adjusted to 1.5 using hydrochloric acid. Then pH is readjusted to 2.5 using sodium hydroxide solution at 10 to 15° C. After precipitation of the product, the white slurry is stirred for 3 hours at 5 to 10° C. Then the precipitate is filtered and is air dried to get Cefdinir D 13.4 g. [HPLC assay 99-99.5%, water content 14.23%]

1. Novel crystalline Form D of cefdinir of the formula (I) 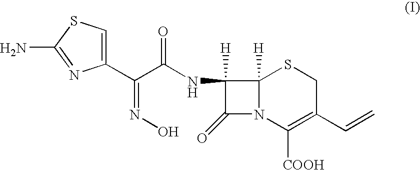
having substantially the same X-ray diffractogram as set out in FIG. 1, and substantially the same IR spectrum, in a KBr, as set out in FIG. 2.

2. The Crystalline Cefdinir Form D of claim 1, which exhibits a characteristic X-ray powder diffraction pattern with characteristic peaks of following 2θ values:

| 2-Theta | I/Iₒ |
|---|---|
| 5.301 | 8.36 |
| 8.399 | 8.27 |
| 10.619 | 100 |
| 11.859 | 18.99 |
| 14.101 | 35.03 |
| 15.119 | 12.74 |
| 18.6 | 10.67 |
| 18.98 | 6.97 |
| 21.32 | 24.79 |
| 22.18 | 8.23 |
| 23.701 | 21.46 |
| 23.96 | 12.33 |
| 24.6 | 8.18 |
| 25.819 | 15.17 |
| 27.54 | 32.7 |
| 28.36 | 13.89 |
| 28.6 | 17.75 |
| 29.161 | 7.88 |
| 30.519 | 6.15 |
| 32.18 | 5.85 |
| 35.94 | 7.62 |
| 38.94 | 7.21 |

3. Crystalline substance of claim 1, which is characterized by infrared absorption spectrum pattern having characteristic peaks at approximately 3301.4, 2980.9, 2898.4, 1786.1, 1760.2, 1667.4, 1610.1, 1544.4, 1458.2, 1429.3, 1419.5, 1349.3, 1334.4, 1299.1, 1285.9, 1191.2, 1136.0, 1121.8, 1064.2, 1049.7, 1016.1, and 993.1 Cm⁻¹.
